(19) United States
(12) Patent Application Publication
(10) Pub. No.: US 2026/0215659 A1
(43) Pub. Date: Jul. 30, 2026

(54) DISPLAY DEVICE

(71) Applicant: NEC Corporation, Tokyo (JP)

(72) Inventors: Shimpei GODA, Tokyo (JP); Takuma IGARASHI, Tokyo (JP)

(73) Assignee: NEC Corporation, Tokyo (JP)

(21) Appl. No.: 19/145,975

(22) PCT Filed: Mar. 28, 2023
(86) PCT No.: PCT/JP2023/012638

§ 371 Date: Jul. 7, 2025

(51) Int. Cl. A61B 1/00; A61B 1/045

(52) U.S. Cl. CPC A61B 1/00009; A61B 1/0002; A61B 1/045

(57) ABSTRACT

A display device includes: a storage device that stores positional information of an endoscope and a score calculated based on image data acquired by the endoscope in such a manner as to be associated; and a display unit that displays the score in time series for each predetermined area corresponding to the positional information, based on the positional information and the score stored in the storage device.

### TECHNICAL FIELD

The present invention relates to a display device, a display method, and a recording medium.

### BACKGROUND ART

There is a known technique for assisting an endoscopic examination.

For example, Patent Literature 1 discloses a system for enabling a medical team to confirm and consider in real time in the middle of an endoscopic examination. According to Patent Literature 1, when a site suspected as lesion is contained in a video frame, the system calculates the positional coordinates of the site suspected as lesion. Moreover, the system generates display information including the presence or absence of a site suspected as lesion and the positional coordinates of the site suspected as lesion. Then, the user's display shows the site suspected as lesion in such a manner as to be visually distinguished based on the display information on the video frame, and shows the positional coordinates of the site suspected as lesion in such a manner as to be visually linked to the site suspected as lesion.

### CITATION LIST

### Patent Literature

Patent Literature 1: Japanese Unexamined Patent Application Publication No. JP-A 2022-145658

### SUMMARY OF INVENTION

### Technical Problem

There is a case where it is desired not only to confirm an AI score calculated in an endoscopic examination during the examination, but also to review it after the examination. However, in the case of the technique as described in Patent Literature 1, it is not assumed to review the calculated AI score after the examination. Therefore, it is difficult to properly review the AI score, and it may be difficult to utilize the AI score after the examination. Thus, there has been a problem that it may be difficult to review an analysis result after an endoscopic examination.

Accordingly, an object of the present invention is to provide a display device, a display method and a recording medium that can solve the abovementioned problem.

### Solution to Problem

In order to achieve the object, a display device as an aspect of the present disclosure includes: a storage device configured to store positional information of an endoscope and a score calculated based on image data acquired by the endoscope in such a manner as to be associated; and a display unit configured to display the score in time series for each predetermined area corresponding to the positional information, based on the positional information and the score stored in the storage device.

Further, a display method as another aspect of the present disclosure is a display method by an information processing device including a storage device storing positional information of an endoscope and a score calculated based on image data acquired by the endoscope in such a manner as to be associated. The display method includes receiving an instruction to display and, in response to the instruction, displaying the score in time series for each predetermined area corresponding to the positional information, based on the positional information and the score stored in the storage device.

Further, a non-transitory computer-readable recording medium as an aspect of the present invention has a program recorded thereon. The program includes instructions for causing an information processing device including a storage device storing positional information of an endoscope and a score calculated based on image data acquired by the endoscope in such a manner as to be associated to receive an instruction to display and, in response to the instruction, display the score in time series for each predetermined area corresponding to the positional information, based on the positional information and the score stored in the storage device.

### Advantageous Effects of Invention

According to the configurations as described above, it is possible to solve the problem as mentioned above.

### BRIEF DESCRIPTION OF THE DRAWINGS

FIG. 1 is a block diagram showing an example of a configuration of a display device in a first example embodiment of the present disclosure.

FIG. 2 is a diagram showing an example of position and score information.

FIG. 3 is a diagram for describing an example of processing by a positional information acquiring unit.

FIG. 4 is a diagram for describing an example of display by a display unit.

FIG. 5 is a flowchart showing an example of operation of the display device.

FIG. 6 is a flowchart showing an example of operation of the display device.

FIG. 7 is a block diagram showing another example of the configuration of the display device.

FIG. 8 is a diagram for describing another example of the display by the display unit.

FIG. 9 is a diagram showing an example of a hardware configuration of a display device in a second example embodiment of the present disclosure.

FIG. 10 is a block diagram showing an example of a configuration of the display device.

### EXAMPLE EMBODIMENT

### First Example Embodiment

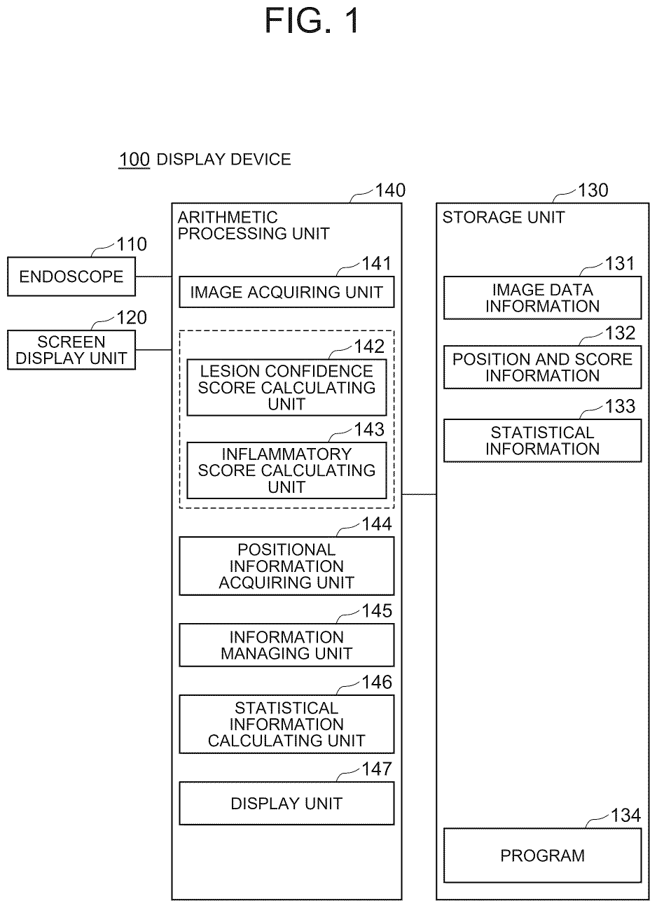

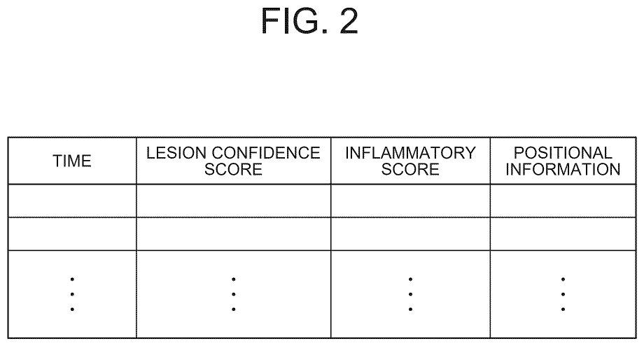

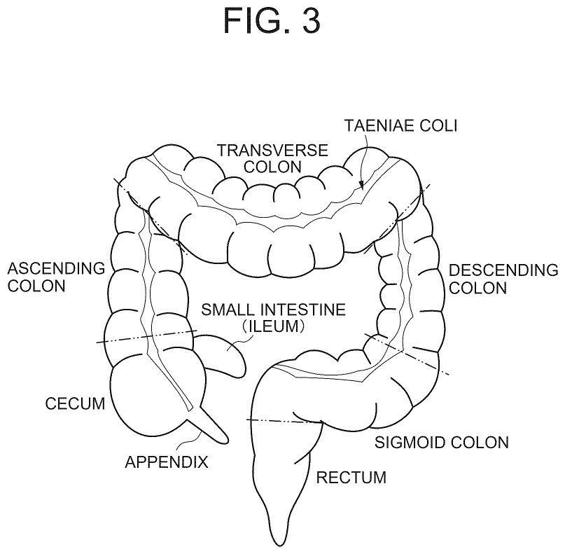

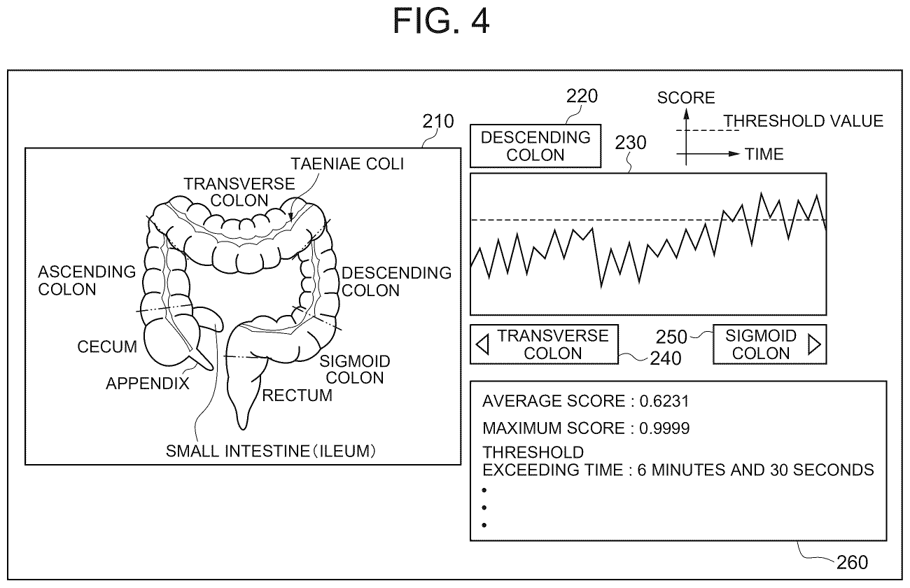

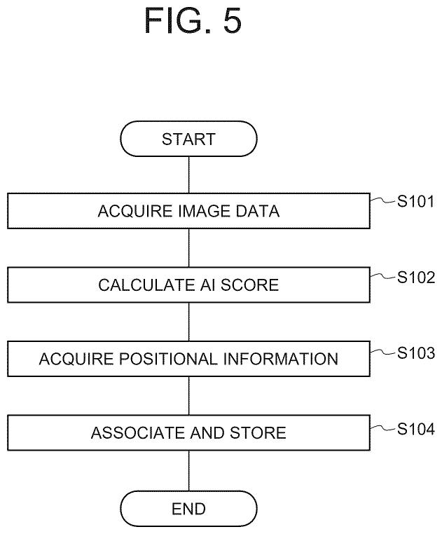

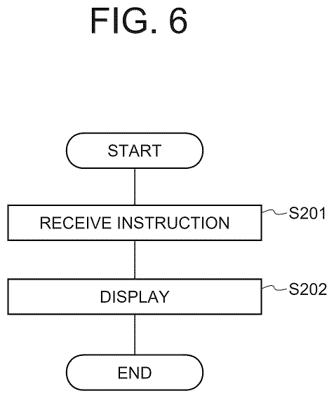

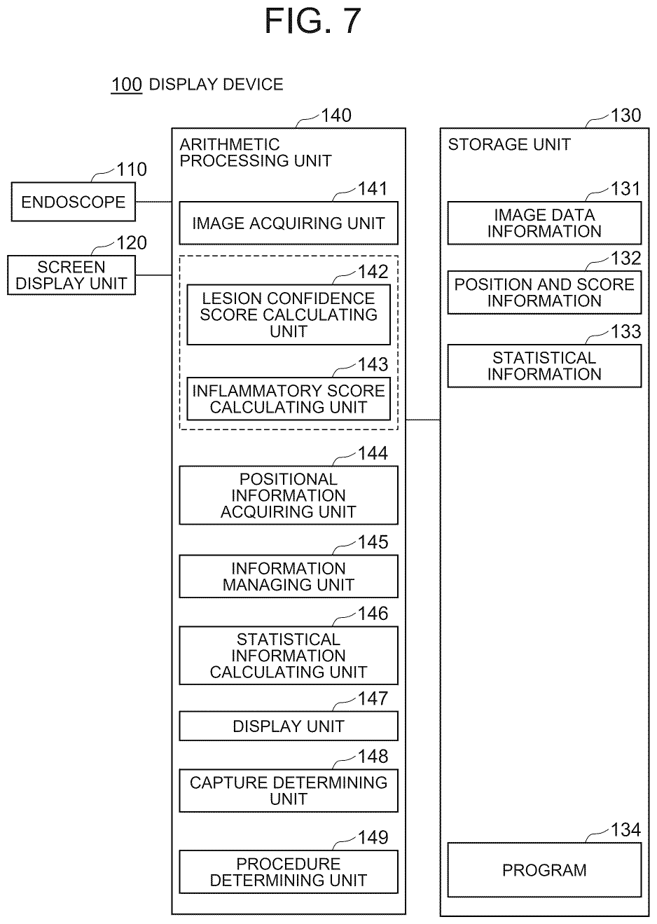

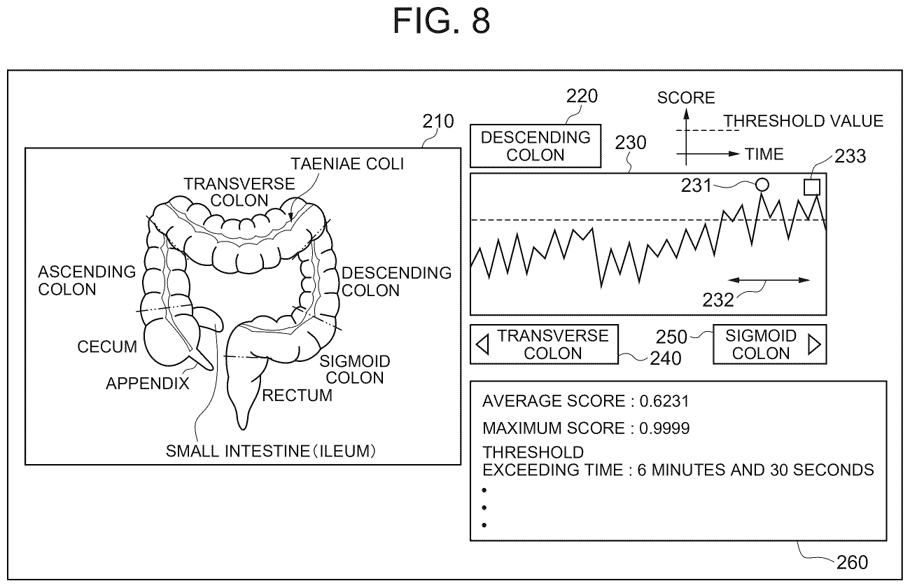

A first example embodiment of the present invention will be described with reference to FIGS. 1 to 8. FIG. 1 is a block diagram showing an example of a configuration of a display device 100. FIG. 2 is a diagram showing an example of position and score information 132. FIG. 3 is a diagram for describing an example of processing by a positional information acquiring unit 144. FIG. 4 is a diagram for describing an example of display by a display unit 147. FIGS. 5 and 6 are flowcharts showing an example of operation of the display device 100. FIG. 7 is a block diagram showing another example of the configuration of the display device 100. FIG. 8 is a diagram for describing another example of the display by the display unit 147.

In the first example embodiment of the present disclosure, the display device 100 that allows review of a visualized analysis result at any timing will be described. As will be described later, the display device 100 stores an AI (Artificial Intelligence) score calculated based on image data acquired from an endoscope 110 and positional information of the endoscope in such a manner as to be associated. Moreover, when displaying, the display device 100 displays a time-series AI score for each area of the large intestine corresponding to the positional information based on the stored positional information and AI score. Consequently, the display device 100 allows review of the time-series AI score for each area.

In the present disclosure, the AI score refers to a score that can be acquired by, for example, inputting image data acquired from the endoscope 110 into a trained model. For example, the AI score indicates a value corresponding to whether or not there is a problem at a site indicated by the image data. As an example, the AI score includes a lesion confidence score indicating whether or not there is a lesion and an inflammatory score such as an inflammatory bowel disease (IBD) score that is a score for measuring the degree of inflammation in the large intestine. As will be described later, the AI score may include size information indicating the size of a lesion, qualitative information indicating the type of a lesion, and the like, and procedure information indicating whether or not a procedure is in progress.

The display device 100 is an information processing device that is connected to the endoscope 110 and so forth and performs the AI score calculation based on the image data acquired from the endoscope 110, and so forth. Moreover, the display device 100 has a function of managing the calculated AI score and the like in such a manner that a visualized analysis result can be reviewed at any timing such as after an endoscopic examination. FIG. 1 shows an example of a configuration of the display device 100. Referring to FIG. 1, the display device 100 includes, as main components, the endoscope 110, a screen display unit 120, a storage unit 130, and an arithmetic processing unit 140, for example.

FIG. 1 illustrates a case of enabling a function as the display device 100 using a single information processing device. However, at least part of the function as the display device 100 may be enabled using a plurality of information processing devices, such as being enabled on the cloud, for example. Moreover, the display device 100 may have a configuration other than the configuration illustrated above, such as an operation input unit composed of an operation input device such as a keyboard and a mouse, or may not include part of the configuration illustrated above.

The endoscope 110 has an optical system such as a small-sized image capture device at an end portion, and acquires image data of the interior of a human body in a state where the end portion is inserted into the interior of the human body. For example, the endoscope 110 acquires a plurality of image data in each area while being withdrawn after being inserted from the anus to the cecum portion. As an example, the endoscope 110 can acquire a plurality of image data of each area in order of the cecum, ascending colon, transverse colon, descending colon, sigmoid colon, and rectum, respectively.

In the present disclosure, the configuration of the endoscope 110 is not specifically limited. The endoscope 110 may be a general one.

The screen display unit 120 is configured with a screen display device such as a liquid crystal display or an organic EL (electro-luminescence). The screen display unit 120 can display on a screen a variety of information and so forth stored in the storage unit 130 in accordance with an instruction from the arithmetic processing unit 140.

The storage unit 130 is a storage device such as a hard disk and memory. The storage unit 130 stores processing information and a program 134 necessary for a variety of processing by the arithmetic processing unit 140. The program 134 enables various processing units by being loaded and executed by the arithmetic processing unit 140. The program 134 is previously loaded from an external device or a recording medium via a data input/output function such as a communication I/F unit, and is stored in the storage unit 130. Main information stored in the storage unit 130 includes, for example, image data information 131, position and score information 132, statistical information 133, and the like.

The image data information 131 includes image data acquired from the endoscope 110. The image data information 131 may include time-series image data acquired from the endoscope 110. For example, in the image data information 131, the time of acquisition of image data and the image data are associated. The image data information 131 is updated, for example, in accordance with acquisition of image data by the image acquiring unit 141 from the endoscope 110.

The position and score information 132 includes AI scores calculated by AI score calculating units such as a lesion confidence score calculating unit 142 and an inflammatory score calculating unit 143, which will be described later, and positional information acquired by a positional information acquiring unit 144. For example, the position and score information 132 is updated, for example, in accordance with storing the AI score in association with the positional information into the storage unit 130 by an information managing unit 145 to be described later.

FIG. 2 shows an example of the position and score information 132. Referring to FIG. 2, in the position and score information 132, the time, a lesion confidence score, an inflammatory score, and positional information are associated. Here, the time indicates, for example, the time of acquisition of the AI score and the positional information. For example, the time may be the time of acquisition of image data used when acquiring the AI score and the positional information. The time may be, for example, an elapsed time from the start of acquisition of image data by the endoscope 110. Moreover, the lesion confidence score is a value indicating whether or not there is a lesion. For example, the lesion confidence score indicates that a larger value is more likely to result in a lesion. As will be described later, the lesion confidence score is calculated based on the image data by the lesion confidence score calculating unit 142. Moreover, the inflammatory score indicates a value such as the IBD score that is a score for measuring the degree of inflammation in the large intestine. For example, the inflammatory score indicates that the greater the value, the greater the degree of inflammation. As will be described later, the inflammatory score is calculated based on the image data by the inflammatory score calculating unit 143. Moreover, the positional information indicates a position where the image data is acquired. For example, the positional information indicates an area in the large intestine where the endoscope 110 is located at the time of acquisition of the image data, such as cecum, the ascending colon, or transverse colon. As will be described later, the positional information is acquired based on the image data and the like by the positional information acquiring unit 144. In the position and score information 132, any information other than those illustrated above may be associated with the time and the like.

The statistical information 133 indicates a statistical value of the AI score for each area or for the entire large intestine. For example, the statistical information 133 includes the average value and maximum value of lesion confidence score and inflammation score, and a threshold exceeding time indicating a time that the value exceeds a threshold value. The statistical information 133 may include a statistical value other than those illustrated above. The statistical information 133 is updated in accordance with calculation of the statistical information by a statistical information calculating unit 146, which will be described later.

The arithmetic processing unit 140 includes an arithmetic logic unit such as a CPU (Central Processing Unit) and a peripheral circuit thereof. The arithmetic processing unit 140 reads the program 134 from the storage unit 130 and executes it, thereby making the above hardware and the program 134 cooperate and enables various processing units. Main processing units enabled by the arithmetic processing unit 140 include, for example, an image acquiring unit 141, a lesion confidence score calculating unit 142, an inflammatory score calculating unit 143, a positional information acquiring unit 144, an information managing unit 145, a statistical information calculating unit 146, and a display unit 147.

The arithmetic processing unit 140 may have a GPU (Graphic Processing Unit), a DSP (Digital Signal Processor), an MPU (Micro Processing Unit), an FPU (Floating point number Processing Unit), a PPU (Physics Processing Unit), a TPU (Tensor Processing Unit), a quantum processor, a microcontroller, or a combination thereof, instead of the abovementioned CPU.

The image acquiring unit 141 acquires image data acquired by the endoscope 110 from the endoscope 110. Moreover, the image acquiring unit 141 stores the acquired image data as the image data information 131 into the storage unit 130. For example, the image acquiring unit 141 may store the image data in association with the time at which the endoscope 110 acquires the image data into the storage unit 130.

The lesion confidence score calculating unit 142 calculates a lesion confidence score, which is a value indicating whether or not there is a lesion, based on the image data acquired by the image acquiring unit 141. In other words, the lesion confidence score calculating unit 142 functions as an AI score calculating unit that calculates the lesion confidence score that is the AI score based on the image data.

For example, the lesion confidence score calculating unit 142 has a model trained to output a lesion confidence score in response to input of the image data by performing machine learning using image data with a label such as presence or absence of a lesion as training data. By input of the image data into the trained model as described above, the lesion confidence score calculating unit 142 can calculate the lesion confidence score corresponding to the image data. The lesion confidence score calculating unit 142 may calculate the lesion confidence score based on the image data by a method other than that illustrated above.

The inflammatory score calculating unit 143 calculates an inflammatory score such as the IBD score that is a score for measuring the degree of inflammation in the large intestine based on the image data acquired by the image acquiring unit 141. In other words, the inflammatory score calculating unit 143 functions as an AI score calculating unit that calculates the inflammatory score that is the AI score based on the image data.

For example, the inflammatory score calculating unit 143 has a model trained to output an inflammatory score in response to input of the image data by performing machine learning using image data provided with a label such as a degree of inflammation as teacher data. The inflammatory score calculating unit 143 can calculate the inflammatory score corresponding to the image data by input of the image data into the trained model as described above. The inflammatory score calculating unit 143 may calculate the inflammatory score based on the image data by a method other than that illustrated above.

The positional information acquiring unit 144 acquires positional information indicating the position of the endoscope 110 at the time the image data is acquired. For example, the positional information acquiring unit 144 acquires information indicating an area of the large intestine where the endoscope 110 is located at the time the image data is acquired, such as the cecum, the ascending colon, the transverse colon and the descending colon, as the positional information.

For example, the positional information acquiring unit 144 can acquire the positional information based on the image data. As an example, the positional information acquiring unit 144 has a model that identifies a predefined landmark such as the ileocecal valve, the hepatic flexure, and the splenic flexure in response to input of the image data. The positional information acquiring unit 144 identifies a landmark in the image data by input of the image data into the trained model as described above. Then, the positional information acquiring unit 144 acquires the positional information in accordance with the identification result. For example, as described above, the endoscope 110 starts acquiring the image data from the cecum portion. After that, the positional information acquiring unit 144 determines whether the position where the image data is acquired is on the cecum side or the ascending colon side in accordance with whether the ileocecal valve, which is a landmark, can be identified in the image data. As a result, the positional information acquiring unit 144 can acquire the positional information corresponding to the identification result. That is to say, the positional information acquiring unit 144 can acquire any of the positional information indicating that it is located in an area called the cecum and the positional information indicating that it is located in an area called the ascending colon in accordance with the identification result. Further, by identifying the hepatic flexure that is a landmark based on the image data, the positional information acquiring unit 144 can identify whether the endoscope 110 has moved from the ascending colon to the transverse colon. For example, as described above, the positional information acquiring unit 144 can acquire the positional information corresponding to the identification result by identifying a predetermined landmark. The positional information acquiring unit 144 may be configured to identify a landmark other than those described above. Further, the model described above may be previously trained by, for example, performing machine learning using image data provided with a label for each landmark as training data.

As illustrated in FIG. 3, the positional information acquiring unit 144 can acquire the positional information indicating which area it is located in among areas such as the cecum, the ascending colon, the transverse colon, the descending colon, the sigmoid colon, and the rectum. The positional information acquiring unit 144 may acquire the positional information indicating which area it is located in among the more subdivided or integrated areas. For example, the positional information acquiring unit 144 may acquire the positional information indicating which area it is located in among the three areas of the ascending colon side, transverse colon, and descending colon side. The positional information acquiring unit 144 may acquire the positional information indicating areas other than those illustrated above.

In a case where the endoscope 110 can acquire shape information such as UPD (Endoscope Position Detecting Unit) information, the positional information acquiring unit 144 may acquire the positional information based on the abovementioned shape information and the like. In other words, the positional information acquiring unit 144 may be configured to acquire the positional information corresponding to the identification result by identifying an area where the endoscope 110 is located, based on the shape information or the like of the endoscope 110. In this manner, the positional information acquiring unit 144 may acquire the positional information based on other than the image data.

The information managing unit 145 associates the AI scores calculated by the lesion confidence score calculating unit 142 and the inflammatory score calculating unit 143 and the positional information acquired by the positional information acquiring unit 144. For example, the information managing unit 145 associates the AI scores and the positional information based on the time. Then, the information managing unit 145 stores the result of association as the position and score information 132 into the storage unit 130.

By performing the above association, the information managing unit 145 can store the position and score information 132 as illustrated in FIG. 2 into the storage unit 130. In other words, by performing the above association, the information managing unit 145 can store the position and score information 132 in which the lesion confidence score, the inflammation score, and the positional information are associated for each time in the memory unit 130.

The statistical information calculating unit 146 calculates a statistical value of the AI score for each area or for the entire large intestine based on the position and score information 132. For example, the statistical information calculating unit 146 calculates, as the statistical value, the average value, maximum value, threshold exceeding time or the like of the lesion confidence score or the inflammation score, which are AI scores. The statistical information calculating unit 146 may calculate the statistical value other than that illustrated above. Further, the statistical information calculating unit 146 stores the calculated statistical value as the statistical information 133 into the storage unit 130.

The display unit 147 displays the position and score information 132, the statistical information 133 and so forth on the screen display unit 120. For example, the display unit 147 displays the position and score information 132, the statistical information 133 and so forth on the screen display unit 120 in accordance with an instruction from an operator or the like of the display device 100. The display unit 147 may display at any timing in accordance with the instruction from the operator or the like.

FIG. 4 shows an example of display by the display unit 147. Referring to FIG. 4, as an example, the display unit 147 can display, on the screen display unit 120, an organ display region 210, an area display region 220, a score display region 230, a previous area display region 240, a next area display region 250, a statistical information display region 260, and so forth. FIG. 4 shows an example of display of each region, and the location where each region is displayed and the size of each region may be adjusted as necessary. Further, the display unit 147 may display only some of the illustrated regions, for example, may display the regions other than the organ display region 210 illustrated in FIG. 4

The organ display region 210 is a region for displaying an outline diagram of the large intestine. For example, in the organ display region 210, the view of the large intestine can be displayed in such a manner that an area being displayed in the score display region 230 can be displayed. As an example, in the organ display region 210, any highlighting may be performed, such as changing the color of an area being displayed in the score display region 230.

Further, the area display region 220 is a region for displaying information indicating an area being displayed in the score display region 230. For example, in the case illustrated in FIG. 4, the area display region 220 indicates that an area being displayed in the score display region 230 is the descending colon. Moreover, the score display region 230 is a region for displaying time-series AI score in any area. In the score display region 230, the lesion confidence score and the inflammatory score that are the AI scores can be displayed in time series in such a manner as to be displayed in different colors and lines, respectively. For example, in the case illustrated in FIG. 4, a larger value of the Y axis indicates a larger value of an AI score. Moreover, in FIG. 4, a predetermined threshold value is superimposed on the display, and a portion that exceeds the threshold value is a portion estimated to be problematic. For example, in the case of FIG. 4, it is highly possible that there is a lesion or inflammation in the portion of the descending colon on the sigmoid side. Meanwhile, only one of the lesion confidence score and the inflammatory score that are AI scores may be displayed or both may be displayed in the score display region 230. The type of the AI score to be displayed in the score display region 230 may be configured to be switchable by any method in accordance with, for example, an instruction from the operator of the display device 100. The previous area display region 240 is a region for displaying information indicating an area one before the area being displayed in the score display region 230. Further, the next area display region 250 is a region for displaying information indicating an area one behind the area being displayed in the score display region 230. For example, the area to be displayed in the score display region 230 may be switched by the operator who operates the display device 100 clicking on the previous area display region 240 or the next area display region 250.

Further, the statistical information display region 260 is a region for displaying information contained in the statistical information 133. For example, the statistical information display region 260 displays the average value, maximum value, threshold exceeding time or the like of the lesion confidence score and the inflammation score. A statistical value other than those illustrated above may be displayed in the statistical information display region 260. The statistical information display region 260 may display a statistical value for each area, or may display a statistical value for a plurality of areas or the entire large intestine. It may be configured so that which statistical value among the statistical values for each area and other statistical values is displayed can be switched by any method in accordance with an instruction from the operator on the display device 100.

The above is an example of the configuration of the display device 100. Subsequently, an example of operation of the display device 100 will be described with reference to FIGS. 5 and 6.

FIG. 5 is a flowchart showing an example of operation of the display device 100 at the time of storing information. Referring to FIG. 5, the image acquiring unit 141 acquires image data acquired by the endoscope 110 from the endoscope 110 (step S101).

The AI score calculating unit calculates an AI score based on the image data (step S102). For example, the lesion confidence score calculating unit 142 calculates a lesion confidence score, which is a value indicating whether or not there is a lesion, based on the image data acquired by the image acquiring unit 141. Further, the inflammatory score calculating unit 143 calculates an inflammatory score such as the IBD score that is a score for measuring the degree of inflammation in the large intestine based on the image data acquired by the image acquiring unit 141. For example, the lesion confidence score calculating unit 142 and the inflammatory score calculating unit 143 may operate in parallel.

The positional information acquiring unit 144 acquires positional information indicating the position of the endoscope 110 when the image data is acquired (step S103). For example, the positional information acquiring unit 144 can acquire the positional information based on the image data.

The information managing unit 145 stores the AI scores calculated by the lesion confidence score calculating unit 142 and the inflammatory score calculating unit 143 and the positional information acquired by the positional information acquiring unit 144 into the storage unit 130 in such a manner as to be associated (step S104). For example, the information managing unit 145 can associate the AI scores and the positional information based on the time.

The above is an example of the operation of the display device 100. Subsequently, an example of operation of the display device 100 when displaying the AI scores and the like will be described with reference to FIG. 6.

FIG. 6 is a flowchart showing an example of the operation of the display device 100 when displaying the AI scores and the like. Referring to FIG. 6, the display unit 147 receives an instruction to display information from an operator of the display device 100 (step S201). In accordance with this, the display unit 147 displays, on the screen display unit 120, the position and score information 132, the statistical information 133 and so forth (step S202). For example, the display unit 147 can perform the display as illustrated in FIG. 4

The above is an example of the operation of the display device 100 when displaying the AI scores and the like.

Thus, the display device 100 has the information managing unit 145 and the display unit 147. According to such a configuration, the display unit 147 can display in time series the AI scores for each area in the large intestine corresponding to the positional information, based on the position and score information stored by the information managing unit 145. As a result, it becomes possible to review the calculated AI scores and the like at any timing such as after an examination in an easy-to-understand manner, and it is possible to assist a doctor in making optimal decision making.

The configuration of the display device 100 is not limited to the case illustrated in FIG. 1. For example, referring to FIG. 7, the arithmetic processing unit 140 loads and executes the program 134, thereby enabling a capture determining unit 148 and a procedure determining unit 149 in addition to the configuration illustrated in FIG. 1.

The capture determining unit 148 determines based on the image data information 131 that a doctor who conducts an examination has captured an image. For example, when the doctor captures an image, the image freezes for a predetermined time. Therefore, in a case where it is determined that the image is freezing for a predetermined time based on time-series image data included in the image data information 131, the capture determining unit 148 determines that the doctor has captured at the corresponding time. Further, the capture determining unit 148 can store information corresponding to the determination result as the position and score information 132 into the storage unit 130. The capture determining unit 148 may determine that the doctor has captured by a method other than that illustrated above.

In a case where the doctor has captured, it is assumed to be highly possible that there is a lesion such as a tumor in image data acquired by the capture. Therefore, the display device 100 may be configured to run a model for measuring the size of a lesion or a model for determining the type of a lesion in accordance with the result of determination by the capture determining unit 148, and acquire information indicating the size of the lesion or the type of the lesion. Further, the information indicating the size of the lesion or the type of the lesion may be stored in the storage unit 130 as the position and score information 132. The model for measuring the size of a lesion and the model for determining the type of a lesion may be, for example, previously trained by machine learning using prepared training data.

The procedure determining unit 149 determines based on the image data information 131 that the doctor has performed some kind of procedure such as excision of the polyp. For example, the procedure determining unit 149 has a model for detecting an instrument used when performing a procedure in image data. The procedure determining unit 149 can determine that the doctor has performed a procedure in accordance with the result of input of image data into the model. For example, in a case where an instrument is detected in the image data, the procedure determining unit 149 may determine that the doctor is performing a procedure. Further, the procedure determining unit 149 can store information corresponding to the determination result as the position and score information 132 into the storage unit 130. The model for detecting an instrument may be, for example, previously trained by machine learning using prepared training data.

FIG. 8 shows another example of the display by the display unit 147. For example, in a case where the display device 100 includes the capture determining unit 148 and the procedure determining unit 149, the display unit 147 can display a capture point 231 and procedure time information 232 on the score display region 230. Further, the display unit 147 can display an image point 233 and the like on the score display region 230 regardless of whether or not the capture determining unit 148 and the procedure determining unit 149 are included.

The capture point 231 indicates the time at which the capture determining unit 148 determines that the doctor has captured. For example, the display unit 147 can display the capture point 231 on a spot corresponding to the time at which it is determined that the doctor has captured in time-series data displayed on the score display region 230. The capture point 231 may be configured in such a manner as to be able to display image data at the time in accordance with any operation on the capture point 231. Further, as described above, the model for measuring the size of a lesion and the model for determining the type of a lesion can be run in accordance with the result of determination by the capture determining unit 148. Therefore, the capture point 231 may be configured in such a manner as to be able to display information indicating the size of a lesion or the type of a lesion in accordance with any operation on the capture point 231. Further, the procedure time information 232 is information indicating a time period in which the procedure determined by the procedure determining unit 149 is being conducted. For example, the display unit 147 can display the procedure time information 232 for a time period in which it is determined that the procedure is being performed in the time-series data displayed on the score display region 230. Moreover, the image point 233 indicates the time at which the image data can be displayed. For example, the display unit 147 may display the image point 233 on the score capture point region 230 in a case where a predetermined condition is satisfied, such as a case where the value of the AI score is equal to or more than a predetermined value at a time other than the capture point 231.

For example, as illustrated above, the display unit 147 can display a variety of information such as the capture point 231 on the score display region 230. The display unit 147 may display, on the score display region 230, information corresponding to a doctor's medical examination and the like other than those illustrated above.

In this example embodiment, a case where the display device 100 displays the result of a large intestine endoscopy has been illustrated. However, the display device 100 may be used to, for example, display the result of an upper digestive endoscopic examination.

### Second Example Embodiment

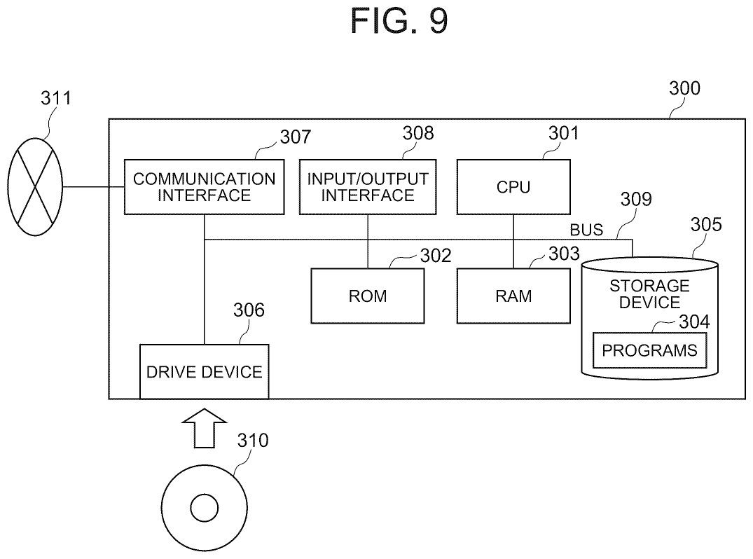

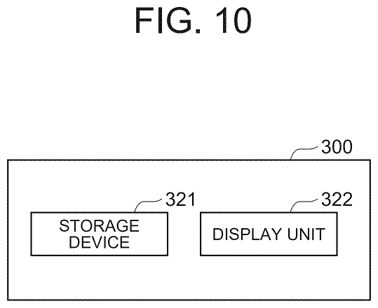

Next, a second example embodiment of the present disclosure will be described with reference to FIGS. 9 and 10. FIG. 9 is a diagram showing an example of a hardware configuration of a display device 300. FIG. 10 is a block diagram showing an example of a configuration of the display device 300.

In the second example embodiment of the present disclosure, the display device 300 including a storage device 321 will be described. FIG. 9 shows an example of the hardware configuration of the display device 300. Referring to FIG. 9, the display device 300 has, as an example, the following hardware configuration including:
a CPU (Central Processing Unit) 301 (arithmetic logic unit);
a ROM (Read Only Memory) 302 (memory unit);
a RAM (Random Access Memory) 303 (memory unit);
programs 304 loaded to the RAM 303;
a storage device 305 storing the programs 304;
a drive device 306 that performs reading from and writing into a recording medium 310 external to an information processing device;
a communication interface 307 connected to a communication network 311 external to the information processing device;
an input/output interface 308 that performs input/output of data; and
a bus 309 connecting the components.

Further, the display device 300 can enable a function as a display unit 322 shown in FIG. 10 by acquisition and execution of the programs 304 by the CPU 301. The programs 304 are, for example, stored in advance in the storage device 305 or the ROM 302 and loaded to the RAM 303 or the like and executed by the CPU 301 as necessary. Moreover, the programs 304 may be provided to the CPU 301 via the communication network 311, or the programs may be stored in advance in the recording medium 310 and read out by the drive device 306 and provided to the CPU 301.

FIG. 9 shows an example of the hardware configuration of the display device 300. The hardware configuration of the display device 300 is not limited to the abovementioned case. For example, the display device 300 may be configured with part of the abovementioned configuration, such as excluding the drive device 306. Further, the CPU 301 may be a GPU or the like illustrated in the first example embodiment.

The storage device 321 stores positional information of an endoscope and a score calculated based on image data acquired by the endoscope in such a manner as to be associated.

The display unit 322 displays in time series a score for each predetermined area corresponding to the positional information, based on the positional information and the score that are stored in the storage device 321.

Thus, the display device 300 includes the storage device 321 and the display unit 322. According to such a configuration, the display unit 322 can display in time series a score for each predetermined area corresponding to the positional information, based on the positional information and the score that are stored in the storage device 321. As a result, it becomes possible to review the score and so forth in an easy-to-understand manner at any timing such as after the examination.

The display device 300 described above can be enabled by incorporation of a predetermined program into an information processing device such as the display device 300. To be specific, a program as another aspect of the present invention is a program for causing an information processing device, such as the display apparatus 300 having the storage device 321 storing position information of an endoscope and a score calculated based on image data acquired by the endoscope in such a manner as to be associated, to receive an instruction to display and, in response to the instruction, display in time series the score for each predetermined area corresponding to the positional information based on the positional information and the score that are stored in the storage device.

Further, the display method performed by an information processing apparatus such as the display device 300 described above receives an instruction to perform a display, and in response to the instruction, causes the score to be displayed in time series for each predetermined area corresponding to the positional information based on the positional information stored in the storage device and the score.

Even if the invention of a program, or a computer-readable recording medium with a program recorded, or a display method has the above configuration, the object of the present disclosure described above can be achieved because the same action and effect as the display device 300 described above are achieved.

### Supplementary Notes

The whole or part of the example embodiments disclosed above can be described as the following supplementary notes. Hereinafter, the overview of the display device and so forth of the present invention will be described. However, the present invention is not limited to the following configurations.

### Supplementary Note 1

A display device comprising:
a storage device configured to store positional information of an endoscope and a score calculated based on image data acquired by the endoscope in such a manner as to be associated; and
a display unit configured to display the score in time series for each predetermined area corresponding to the positional information, based on the positional information and the score stored in the storage device.

### Supplementary Note 2

The display device according to supplementary note 1, comprising:
a positional information acquiring unit configured to acquire the positional information;
a score calculating unit configured to calculate, based on the image data acquired by the endoscope, the score that is a value corresponding to whether or not a problem is occurring at a site indicated by the image data; and
a managing unit configured to store, in the storage device, the positional information acquired by the positional information acquiring unit and the score calculated by the score calculating unit in such a manner as to be associated, wherein
the display unit displays the score in time-series for each predetermined area corresponding to the positional information, based on the position information and the score stored in the storage device by the managing unit.

### Supplementary Note 3

The display device according to supplementary note 2, wherein
the positional information acquiring unit acquires the positional information based on the image data acquired by the endoscope.

### Supplementary Note 4

The display device according to supplementary note 3, wherein
the positional information acquiring unit acquires the positional information based on a result of identification of a predetermined landmark in the image data.

### Supplementary Note 5

The display device according to any one of supplementary notes 2 to 4, wherein
the positional information acquiring unit acquires, as the positional information, information indicating an area in large intestine where the endoscope is located when acquiring the image data.

### Supplementary Note 6

The display device according to any one of supplementary notes 2 to 5, wherein:
the score calculating unit calculates, as the score, a lesion confidence score indicating whether or not there is a lesion and an inflammation score that is a score for measuring a degree of inflammation in large intestine; and
the display unit displays indication of the lesion confidence score and the inflammation score in such a manner as to be distinguishable.

### Supplementary Note 7

The display device according to any one of supplementary notes 2 to 6, wherein
the managing unit associates the positional information acquired by the positional information acquiring unit with the score calculated by the score calculating unit based on time.

### Supplementary Note 8

The display device according to any one of supplementary notes 1 to 7, wherein
the display unit displays the score in time series and also displays, in a position corresponding to time satisfying a predetermined condition in a region to display in time series, an image point capable of showing the image data acquired by the endoscope at the time.

### Supplementary Note 9

The display device according to any one of supplementary notes 1 to 8, comprising
a capture determining unit configured to determine that a doctor who conducts an examination has captured an image based on the image data, wherein
the display unit displays the score in time series and also displays a capture point indicating that the doctor has captured the image.

### Supplementary Note 10

The display device according to any one of supplementary note 1 to 9, comprising
a procedure determining unit configured to determine that a doctor has conducted a predetermined procedure based on the image data, wherein
the display unit displays the score in time series and also displays procedure time information indicating a time period during which the doctor has conducted the procedure.

### Supplementary Note 11

The display device according to any one of supplementary notes 1 to 10, comprising
a statistical information calculating unit configured to calculate a statistical value based on the positional information and the score stored, wherein
the display unit displays the score in time series and also displays the statistical value.

### Supplementary Note 12

A display method by an information processing device including a storage device storing positional information of an endoscope and a score calculated based on image data acquired by the endoscope in such a manner as to be associated, the display method comprising
receiving an instruction to display and, in response to the instruction, displaying the score in time series for each predetermined area corresponding to the positional information, based on the positional information and the score stored in the storage device.

### Supplementary Note 13

A program comprising instructions for causing an information processing device including a storage device storing positional information of an endoscope and a score calculated based on image data acquired by the endoscope in such a manner as to be associated to
receive an instruction to display and, in response to the instruction, display the score in time series for each predetermined area corresponding to the positional information, based on the positional information and the score stored in the storage device.

The program described in the above example embodiments and supplementary notes may be stored in a storage device, or the program may be recorded on a computer-readable recording medium. For example, the recording medium is a portable medium such as flexible disk, optical disk, magneto-optical disk, and semiconductor memory.

Although the present invention has been described above with reference to the above example embodiments, the present invention is not limited to the example embodiments described above. The configuration and details of the present invention can be changed in various manners that can be understood by those skilled in the art within the scope of the present invention.

### REFERENCE SIGNS LIST

100 display device
110 endoscope
120 screen display unit
130 storage unit
131 image data information
132 position and score information
133 statistical information
134 program
140 arithmetic processing unit
141 image acquiring unit
142 lesion confidence score calculating unit
143 inflammatory score calculating unit
144 positional information acquiring unit
145 information managing unit
146 statistical information calculating unit
147 display unit
148 capture determining unit
149 procedure determining unit
210 organ display region
220 area display region
230 score display region
231 capture point
232 procedure time information
233 image point
300 display device
301 CPU
302 ROM
303 RAM
304 programs
305 storage device
306 drive device
307 communication interface
308 input/output interface
309 bus
310 recording medium
311 communication network
321 storage device
322 display unit

What is claimed is:

1. A display device comprising:
at least one memory storing processing instructions;
at least one processor configured to execute the processing instructions; and
a storage device configured to store positional information of an endoscope and a score calculated based on image data acquired by the endoscope in such a manner as to be associated, wherein the processor is configured to execute the processing instructions to:
display the score in time series for each predetermined area corresponding to the positional information, based on the positional information and the score stored in the storage device.

2. The display device according to claim 1, wherein the processor is further configured to execute the processing instructions to:
acquire the positional information;
calculate, based on the image data acquired by the endoscope, the score that is a value corresponding to whether or not a problem is occurring at a site indicated by the image data; and
store, in the storage device, the acquired positional information and the calculated score in such a manner as to be associated, and display the score in time-series for each predetermined area corresponding to the positional information, based on the position information and the score stored in the storage device.

3. The display device according to claim 2, wherein the processor is further configured to execute the processing instructions to
acquire the positional information based on the image data acquired by the endoscope.

4. The display device according to claim 3, wherein the processor is further configured to execute the processing instructions to
acquire the positional information based on a result of identification of a predetermined landmark in the image data.

5. The display device according to claim 2, wherein the processor is further configured to execute the processing instructions to
acquire, as the positional information, information indicating an area in large intestine where the endoscope is located when acquiring the image data.

6. The display device according to claim 2, wherein the processor is further configured to execute the processing instructions to:
calculate, as the score, a lesion confidence score indicating whether or not there is a lesion and an inflammation score that is a score for measuring a degree of inflammation in large intestine; and
display indication of the lesion confidence score and the inflammation score in such a manner as to be distinguishable.

7. The display device according to claim 2, wherein the processor is further configured to execute the processing instructions to
associate the acquired positional information with the calculated score based on time.

8. The display device according to claim 1, wherein the processor is further configured to execute the processing instructions to
display the score in time series and also displays, in a position corresponding to time satisfying a predetermined condition in a region to display in time series, an image point capable of showing the image data acquired by the endoscope at the time.

9. The display device according to claim 1, wherein the processor is further configured to execute the processing instructions to:
determine that a doctor who conducts an examination has captured an image based on the image data; and
display the score in time series and also display a capture point indicating that the doctor has captured the image.

10. The display device according to claim 1, wherein the processor is further configured to execute the processing instructions to
determine that a doctor has conducted a predetermined procedure based on the image data; and
display the score in time series and also display procedure time information indicating a time period during which the doctor has conducted the procedure.

11. The display device according to claim 1, wherein the processor is further configured to execute the processing instructions to:
calculate a statistical value based on the positional information and the score stored; and
display the score in time series and also displays the statistical value.

12. A display method by an information processing device including a storage device storing positional information of an endoscope and a score calculated based on image data acquired by the endoscope in such a manner as to be associated, the display method comprising
receiving an instruction to display and, in response to the instruction, displaying the score in time series for each predetermined area corresponding to the positional information, based on the positional information and the score stored in the storage device.

13. A non-transitory computer-readable recording medium having a program recorded thereon, the program comprising instructions for causing an information processing device including a storage device storing positional information of an endoscope and a score calculated based on image data acquired by the endoscope in such a manner as to be associated to
receive an instruction to display and, in response to the instruction, display the score in time series for each predetermined area corresponding to the positional information, based on the positional information and the score stored in the storage device.

14. The display device according to claim 1, wherein the processor is further configured to support medical decision making by displaying the time series AI scores for each area, wherein the AI scores are calculated using machine learning models trained to detect abnormalities in the image data.
